(19) United States
(12) Patent Application Publication
(10) Pub. No.: US 2006/0008921 A1
(43) Pub. Date: Jan. 12, 2006

(54) Immunochromatographic methods for detecting an analyte in a sample which employ semiconductor nanocrystals as detectable labels

(75) Inventors: Robert H. Daniels, Palo Alto, CA (US); Andrew R. Watson, Belmont, CA (US)

Correspondence Address: ROBINS & PASTERNAK, 1731 EMBARCADERO ROAD, SUITE 230, PALO ALTO, CA, 94303, US

(73) Assignee: Quantum Dot Corporation

(21) Appl. No.: 10/911,364

(22) Filed: Aug. 3, 2004

Related U.S. Application Data
(63) Continuation of application No. 09/750,223, filed on Dec. 27, 2000, now abandoned.
(60) Provisional application No. 60/180,811, filed on Feb. 7, 2000.

(51) Int. Cl. G01N 33/558

(52) U.S. Cl. 436/514

(57) ABSTRACT

Immunochromatographic test strip assays which employ semiconductor nanocrystals as detectable labels are disclosed, as are methods for detecting and quantifying one or more analytes of interest in a test sample using those assays. The test strips of the present invention permit detection and quantitation of one or more analytes of interest present in a test sample suspected of containing them, by using more than one semiconductor nanocrystal as a detectable label, each of which emits exhibits a unique emission peak.

### CROSS-REFERENCE TO RELATED APPLICATION

This application is related to U.S. provisional patent application Ser. No. 60/180,811 filed Feb. 7, 2000, from which priority is claimed under 35 U.S.C. § 119(e)(1) and which is incorporated herein by reference in its entirety.

### FIELD OF THE INVENTION

The present invention relates generally to methods and devices for the detection of analytes in a sample. In particular, the invention relates to immunochromatographic test strips that use semiconductor nanocrystals as a detectable label. The invention further relates to immunochromatographic test strips in which multiple analytes can be detected simultaneously by using more than one semiconductor nanocrystal as a detectable label, each of which emits at a distinct wavelength. The invention further relates to immunochromatographic test strips in which one or more analytes can be detected quantitatively.

### BACKGROUND OF THE INVENTION

Immunochromatographic, lateral flow or strip tests are well-established diagnostic tools for detecting the presence of analytes. A wide variety of strip tests exist; among the more commonly known examples are home pregnancy tests, home ovulation predictor tests, and point-of-care tests for Strep throat and Chlamydia infections.

Test strips offer the advantages of a simple, user-friendly format, and rapidly obtained results that are easily interpreted. The tests themselves are stable for long periods in a variety of climates, and are relatively easy and inexpensive to make. These characteristics well suit them for applications such as home testing, rapid point-of-care testing, and field testing for various environmental and agricultural analytes. They are especially beneficial in that they can provide a means of reliable diagnostic testing that might not otherwise be available to persons in third world countries.

The general principle underlying test strip assays is that a ligand bound by a visually detectable solid support can be measured qualitatively (and in some cases semi-quantitatively). Thus, while test strips may employ any one of a variety of assay schemes, including sandwich assays (both direct and indirect) and competitive reaction assays, all have in common the element of a detectable label that permits identification of an analyte of interest when present in an experimental sample.

Radiolabeled molecules and compounds are frequently used as detectable labels; however, due to the inherent problems associated with the use of radioactive isotopes, which include safety and regulatory burdens, nonradioactive labels are often preferred.

Enzymes, the substrates of which undergo a color change following catalysis have also been used as detectable labels. A number of enzymes which act on such chromogenic substrates have been employed in, for example, ELISA assays, including alkaline phosphatase, horseradish peroxidase, and β-galactosidase.

Metal Sol particles have also been used as detectable labels in immunochromatographic strip assays. See Leuvering, U.S. Pat. No. 4,313,734 (issued Feb. 2, 1982). Particles, composed of either metals or metallic compounds, or polymer nuclei coated with metal or metallic compounds, are coated, either partially or completely, with a ligand specific for an analyte of interest. After reacting the sol with a test sample which contains the analyte of interest, the analyte may then be detected by a variety of means. Owing to the fact that metal sols are colored, visual detection of a positive result is possible, and the use of metal sols of different colors permits detection of multiple analytes in a single strip test assay. However, the results obtained are at best only semi-quantitative. Further, long incubation periods (from 1 hour to overnight) of the analyte-containing sample with the labeled specific binding materials used to detect the analyte's presence are required, thereby greatly reducing the convenience of tests using these labels.

Similarly, the use of colloidal particles, both metal and non-metal, have been employed as detectable labels for the purpose of immunoassays. See, e.g., Yost et al., U.S. Pat. No. 4,954,452 (issued Sep. 4, 1990); Ching et al., U.S. Pat. No. 5,120,643 (issued Jun. 9, 1992). However, colloidal particle-labeled specific binding materials are highly susceptible to aggregation, and are therefore not amenable to rapid efficient transport on chromatographic media without the use of selected solvents and chromatographic transport facilitating agents.

Fluorescent molecules are commonly used as tags for detecting an analyte of interest. Fluorescence is the emission of light resulting from the absorption of radiation at one wavelength (excitation) followed by nearly immediate reradiation usually at a different wavelength (emission). Organic fluorescent dyes are typically used in this context. However, there are chemical and physical limitations to the use of such dyes.

One of these limitations is the variation of excitation wavelengths of different colored dyes. As a result, the simultaneous use of two or more fluorescent tags with different excitation wavelengths requires multiple excitation light sources.

Another drawback of organic dyes is the deterioration of fluorescence intensity upon prolonged and/or repeated exposure to excitation light. This fading, called photo-bleaching, is dependent on the intensity of the excitation light and the duration of the illumination. In addition, conversion of the dye into a nonfluorescent species is irreversible. Furthermore, the degradation products of dyes are organic compounds which may interfere with the biological processes being examined.

Additionally, spectral overlap exists from one dye to another. This is due, in part, to the relatively wide emission spectra of organic dyes and the overlap of the spectra near the tailing region. Few low molecular weight dyes have a combination of a large Stokes shift, which is defined as the separation of the absorption and emission maxima, and high fluorescence output. In addition, low molecular weight dyes may be impractical for some applications because they do not provide a bright enough fluorescent signal.

Furthermore, the differences in the chemical properties of standard organic fluorescent dyes make multiple, parallel assays impractical as different chemical reactions may be involved for each dye used in the variety of applications of fluorescent labels.

Therefore, there is a need for test strip assays that are inexpensive to produce, easy to use, and capable of giving quantitative results for multiple analytes.

### SUMMARY OF THE INVENTION

The present invention is based on the discovery that semiconductor nanocrystals and microspheres dyed with semiconductor nanocrystals, can be used as reliable and sensitive detectable labels in a variety of biological and chemical formats, including immunochromatographic strip assays. Semiconductor nanocrystals (also know as quantum dots and Qdot™ nanocrystals) can be produced that have characteristic spectral emissions. These spectral emissions can be tuned to a desired energy by varying the particle size, size distribution and/or composition of the particle. A targeting compound that has affinity for one or more selected biological or chemical targets is associated with the semiconductor nanocrystal. Thus, the semiconductor nanocrystal will interact or associate with the target due to the affinity of the targeting compound for the target. The location and/or nature of the association can be determined, for example, by irradiation of the sample with an energy source, such as an excitation light source. The semiconductor nanocrystal emits a characteristic emission spectrum which can be observed and measured, for example, spectroscopically.

Conveniently, emission spectra of a population of semiconductor nanocrystals can be manipulated to have linewidths as narrow as 25-30 nm, depending on the size distribution heterogeneity of the sample population, and lineshapes that are symmetric, gaussian or nearly gaussian with an absence of a tailing region. Accordingly, the above technology allows for detection of one, or even several, different biological or chemical moieties in a single reaction. The combination of tunability, narrow linewidths, and symmetric emission spectra without a tailing region provides for high resolution of multiply sized nanocrystals, e.g., populations of monodisperse semiconductor nanocrystals having multiple distinct size distributions within a system, and simultaneous detection of a variety of biological moieties.

In addition, the range of excitation wavelengths of such nanocrystals is broad and can be higher in energy than the emission wavelengths of all available semiconductor nanocrystals. Consequently, this allows the use of a single energy source, such as light, usually in the ultraviolet or blue region of the spectrum, to effect simultaneous excitation of all populations of semiconductor nanocrystals in a system having distinct emission spectra. Semiconductor nanocrystals are also more robust than conventional organic fluorescent dyes and are more resistant to photobleaching than the organic dyes. The robustness of the nanocrystal also alleviates the problem of contamination of degradation products of the organic dyes in the system being examined. Therefore, the present invention provides uniquely valuable tags for detection of biological and chemical molecules which are especially advantageous in the context of strip assays.

Accordingly, in one embodiment, the invention is directed to a method for determining the presence and/or amount of an analyte of interest in a test sample. The method comprises the steps of:

(I) applying the test sample to a test strip to form a sample mixture in a sample reservoir, the test strip comprising
(A) a chromatographic medium;
(B) the sample reservoir disposed on the chromatographic medium for receiving the test sample, the sample reservoir comprising
   (i) a first detection reagent comprising
      (a) a first detection ligand capable of selectively binding a first target moiety of the analyte of interest, wherein (i) the first detection ligand is conjugated with a semiconductor nanocrystal which, when exposed to a light of a selected excitation wavelength, is capable of emitting light of a characteristic emission peak, and
   (ii) binding of the first detection ligand to the first target moiety forms a detection complex,
(C) a capture reagent immobilized on the chromatographic medium within a capture region which is distinct from the sample reservoir, wherein the capture reagent comprises a capture ligand capable of selectively binding the first detection complex to form an immobilized capture complex; and
(D) a control ligand immobilized on the chromatographic medium within a control region distinct from the sample reservoir and the capture region, wherein the control ligand is capable of selectively binding the first detection ligand to form an immobilized control complex;
wherein (i) the test strip has first and second ends, the sample reservoir is disposed at the first end, and the capture region is interposed between the sample reservoir and the control region, (ii) the sample mixture comprises the test sample and the first detection reagent, (iii) the sample mixture is transported via the chromatographic medium from the first to the second end, (iv) the first detection ligand binds the first target moiety to form the detection complex, the detection complex is bound by the capture reagent, and the first detection ligand which is not bound to the first target moiety is bound to the control ligand; and

(II) exposing the test strip to the light of a selected excitation wavelength, wherein the production of light of the characteristic emission peak in both the capture and control regions is indicative of the presence of the analyte in the test sample.

In another embodiment, the invention is directed to a method for determining the presence and/or amount of an analyte of interest in a test sample. The method comprises the steps of:

(I) applying the test sample to a test strip to form a sample mixture in a sample reservoir, the test strip comprising
(A) a chromatographic medium;
(B) the sample reservoir disposed on the chromatographic medium for receiving the test sample, the sample reservoir comprising
   (i) a first detection reagent, comprising
      (a) a first detection ligand capable of selectively binding a first target moiety of the analyte of interest,
      wherein the first detection ligand is conjugated with a semiconductor nanocrystal which, when exposed to a light of a selected excitation wavelength, is capable of emitting light of a characteristic emission peak, and
   (ii) a second detection reagent comprising a second detection ligand capable of selectively binding (a) a second target moiety of the analyte of interest, and (b) a capture ligand,
   wherein binding of the first detection ligand to the first target moiety and the second detection ligand to the second target moiety forms a detection complex,
(C) a capture reagent immobilized on the chromatographic medium within a capture region which is distinct from the sample reservoir, wherein the capture reagent comprises a capture ligand capable of selectively binding the second detection ligand to form an immobilized capture complex; and
(D) a control ligand immobilized on the chromatographic medium within a control region distinct from the sample reservoir and the capture region, wherein the control ligand is capable of selectively binding the first detection ligand not bound to the first target moiety to form an immobilized control complex;

wherein (i) the test strip has first and second ends, the sample reservoir is disposed at the first end, and the control region is interposed between the sample reservoir and the capture region, (ii) the sample mixture comprises the test sample and the first detection reagent, (iii) the sample mixture is transported via the chromatographic medium from the first to the second end, (iv) the first detection ligand binds the first target moiety and the second detection ligand binds the second target moiety to form the detection complex, (v) the detection complex is bound by the capture reagent, and (vi) the first detection ligand which is not bound to the first target moiety is bound to the control ligand; and

(II) exposing the test strip to the light of a selected excitation wavelength, wherein the production of light of the characteristic emission peak in both the capture and control regions is indicative of the presence of the analyte in the test sample.

In yet another embodiment, the invention is directed to a method for determining the presence and/or amount of an analyte of interest in a test sample. The method comprises the steps of:

(I) applying the test sample to a test strip to form a sample mixture in a sample reservoir, the test strip comprising
(A) a chromatographic medium;
(B) the sample reservoir disposed on the chromatographic medium for receiving the test sample, the sample reservoir comprising
   (i) a first detection reagent comprising
      (a) a first detection ligand capable of selectively binding a first target moiety of the analyte of interest, wherein (i) the first detection ligand is conjugated with a semiconductor nanocrystal which, when exposed to a light of a selected excitation wavelength, is capable of emitting light of a characteristic emission peak, and
   (ii) binding of the first detection ligand to the first target moiety forms a first detection complex,
(C) a capture reagent immobilized on the chromatographic medium within a capture region which is distinct from the sample reservoir, wherein the capture reagent comprises a capture ligand capable of selectively binding the first detection complex to form an immobilized capture complex; and
(D) a control reagent immobilized on the chromatographic medium within a control region distinct from the sample reservoir and the capture region, wherein the control ligand is capable of selectively binding the first detection ligand to form an immobilized control complex;
wherein (i) the test strip has first and second ends, the sample reservoir is disposed at the first end, and the capture region is interposed between the sample reservoir and the control region, (ii) the sample mixture comprises the test sample and the first detection reagent, (iii) the sample mixture is transported via the chromatographic medium from the first to the second end, (iv) the first detection ligand binds the first target moiety and the second detection ligand binds the second target moiety to form the detection complex, (v) the detection complex is bound by the capture reagent, and (vi) the first detection ligand which is not bound to the first target moiety is bound to the control ligand; and

(II) exposing the test strip to the light of a selected excitation wavelength, wherein the production of light of the characteristic emission peak in both the capture and control regions is indicative of the presence of the analyte in the test sample.

In the methods above, the amount of analyte in the test sample may be quantified by measuring the quantity of light emitted by the capture region.

In another embodiment, the invention is directed to a test strip for determining the presence and/or amount of an analyte of interest suspected of being present in a test sample. The test strip comprises:

(I) a chromatographic medium;

(II) a sample reservoir disposed on the chromatographic medium for receiving the test sample, the sample reservoir comprising
(A) a first detection reagent comprising
   (i) a first detection ligand capable of selectively binding a first target moiety of the analyte of interest, wherein (a) the first detection ligand is conjugated with a semiconductor nanocrystal which, when exposed to a light of a selected excitation wavelength, is capable of emitting light of a characteristic emission peak, and (b) binding of the first detection ligand to the first target moiety forms a first detection complex,

(II) a capture reagent immobilized on the chromatographic medium within a capture region which is distinct from the sample reservoir, wherein the capture reagent comprises a capture ligand capable of selectively binding the first detection complex to form an immobilized capture complex; and

(III) a control ligand immobilized on the chromatographic medium within a control region distinct from the sample reservoir and the capture region, wherein the control ligand is capable of selectively binding the first detection ligand to form an immobilized control complex.

In still a further embodiment, the invention is directed to a test strip for determining the presence and/or amount of an analyte of interest suspected of being present in a test sample. The test strip comprises:

(I) a chromatographic medium;

(II) a sample reservoir disposed on the chromatographic medium for receiving the test sample, the sample reservoir comprising
(A) a first detection reagent comprising
   (i) a first detection ligand capable of selectively binding a first target moiety of the analyte of interest, wherein the first detection ligand is conjugated with a semiconductor nanocrystal which, when exposed to a light of a selected excitation wavelength, is capable of emitting light of a characteristic emission peak,
(B) a second detection reagent comprising
   (i) a second detection ligand capable of selectively binding (a) a second target moiety of the analyte of interest, and (b) a capture ligand,

(III) a capture reagent immobilized on the chromatographic medium within a capture region which is distinct from the sample reservoir, wherein the capture reagent comprises a capture ligand capable of selectively binding the second detection ligand to form an immobilized capture complex; and

(IV) a control ligand immobilized on the chromatographic medium within a control region distinct from the sample reservoir and the capture region, wherein the control ligand is capable of selectively binding the first detection ligand not bound to the first target moiety to form an immobilized control complex, wherein the test strip has first and second ends, the sample reservoir is disposed at the first end, and the capture region is interposed between the sample reservoir and the control region.

These and other embodiments of the present invention will readily occur to those of ordinary skill in the art in view of the disclosure herein.

### BRIEF DESCRIPTION OF THE DRAWINGS

FIG. 1 is a schematic of a direct-type sandwich assay test strip which employs semiconductor nanocrystals as the detectable label.

FIG. 2 is a schematic of a biotin/streptavidin enhanced-type sandwich assay test strip which employs semiconductor nanocrystals as the detectable label.

FIG. 3 is a schematic of a competitive binding-type assay test strip which employs semiconductor nanocrystals as the detectable label.

FIG. 4 is a graphic representation of the results obtained from the test strip assay described in Example 1.

### DETAILED DESCRIPTION OF THE INVENTION

The practice of the present invention will employ, unless otherwise indicated, conventional methods of chemistry, biochemistry, biology, molecular biology, recombinant DNA technology, and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

All publications, patents and patent applications cited herein, whether supra (i.e., above) or infra (i.e., below) are hereby incorporated by reference in their entirety.

### 1. DEFINITIONS

In describing the present invention, the following terms will be employed, and are intended to be defined as indicated below. Unless otherwise indicated, all terms used herein have the same meaning as they would to one skilled in the art of the present invention.

As used in this specification and the appended claims, the singular forms “a,” “an” and “the” include plural references unless the content clearly dictates otherwise. Thus, for example, reference to “an X” includes a mixture of two or more such X's, “a Y” includes more than one such Y, and the like.

The terms “semiconductor nanocrystal,” “quantum dot” and “Qdot™ nanocrystal” are used interchangeably herein and refer to an inorganic crystallite between about 1 nm and about 1000 nm in diameter or any integer or fraction of an integer therebetween, preferably between about 2 nm and about 50 nm or any integer or fraction of an integer therebetween, more preferably about 2 nm to about 20 nm (such as about 2, 3, 4, 5, 6, 7, 8, 9, 10, 11, 12, 13, 14, 15, 16, 17, 18, 19, or 20 nm). A semiconductor nanocrystal is capable of emitting electromagnetic radiation upon excitation (i.e., the semiconductor nanocrystal is luminescent) and includes a “core” of one or more first semiconductor materials, and may be surrounded by a “shell” of a second semiconductor material. A semiconductor nanocrystal core surrounded by a semiconductor shell is referred to as a “core/shell” semiconductor nanocrystal. The surrounding “shell” material will preferably have a bandgap energy that is larger than the bandgap energy of the core material and may be chosen to have an atomic spacing close to that of the “core” substrate. The core and/or the shell can be a semiconductor material including, but not limited to, those of the group II-VI (ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like) materials, and an alloy or a mixture thereof.

A semiconductor nanocrystal is, optionally, surrounded by a “coat” of an organic capping agent. The organic capping agent may be any number of materials, but has an affinity for the semiconductor nanocrystal surface. In general, the capping agent can be an isolated organic molecule, a polymer (or a monomer for a polymerization reaction), an inorganic complex, and an extended crystalline structure. The coat is used to convey solubility, e.g., the ability to disperse a coated semiconductor nanocrystal homogeneously into a chosen solvent, functionality, binding properties, or the like. In addition, the coat can be used to tailor the optical properties of the semiconductor nanocrystal. Methods for producing capped semiconductor nanocrystals are discussed further below.

Thus, the terms “semiconductor nanocrystal,” “quantum dot” and “Qdot™ nanocrystal” as used herein denote a coated semiconductor nanocrystal core, as well as a core/shell semiconductor nanocrystal.

By “luminescence” is meant the process of emitting electromagnetic radiation (light) from an object. Luminescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including chemical, thermal, electrical, magnetic, electromagnetic, physical or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

“Monodisperse particles” include a population of particles wherein at least about 60% of the particles in the population, more preferably 75% to 90% of the particles in the population, or any integer in between this range, fall within a specified particle size range. A population of monodispersed particles deviate less than 10% rms (root-mean-square) in diameter and pr&ferably less than 5% rms.

The phrase “one or more sizes of semiconductor nanocrystals” is used synonymously with the phrase “one or more particle size distributions of semiconductor nanocrystals.” One of ordinary skill in the art will realize that particular sizes of semiconductor nanocrystals are actually obtained as particle size distributions.

By use of the term “a narrow wavelength band” or “narrow spectral linewidth” with regard to the electromagnetic radiation emission of the semiconductor nanocrystal is meant a wavelength band of emissions not exceeding about 40 nm, and preferably not exceeding about 20 nm in width and symmetric about the center, in contrast to the emission bandwidth of about 100 nm for a typical dye molecule with a red tail which may extend the bandwidth out as much as another 100 nm. It should be noted that the bandwidths referred to are determined from measurement of the full width of the emissions at half peak height (FWHM), and are appropriate in the range of 200 nm to 2000 nm.

By use of the term “a broad wavelength band,” with regard to the excitation of the semiconductor nanocrystal is meant absorption of radiation having a wavelength equal to, or shorter than, the wavelength of the onset radiation (the onset radiation is understood to be the longest wavelength (lowest energy) radiation capable of being absorbed by the semiconductor nanocrystal). This onset occurs near to, but at slightly higher energy than the “narrow wavelength band” of the emission. This is in contrast to the “narrow absorption band” of dye molecules which occurs near the emission peak on the high energy side, but drops off rapidly away from that wavelength and is often negligible at wavelengths further than 100 nm from the emission.

The term “emission peak” refers to the wavelength of light within the characteristic emission spectra exhibited by a particular semiconductor nanocrystal size distribution that demonstrates the highest relative intensity.

The term “excitation wavelength” refers to light having a wavelength lower than the emission peak of the semiconductor nanocrystal used in the first detection reagent.

The present invention provides assays which utilize specific binding members. A “specific binding member” (also referred to as a “ligand”), as used herein, is a member of a “specific binding pair” or “binding pair”, terms used to describe two different molecules where one of the molecules, through chemical or physical means, specifically binds to the second molecule. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent. Unless the context clearly indicates otherwise, the terms “affinity molecule” and “target analyte” are also used herein to refer to first and second members of a binding pair, respectively.

Exemplary binding pairs include any haptenic or antigenic compound in combination with a corresponding antibody or binding portion or fragment thereof (e.g., digoxigenin and anti-digoxigenin; fluorescein and anti-fluorescein; dinitrophenol and anti-dinitrophenol; bromodeoxyuridine and anti-bromodeoxyuridine; mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., acetylcholine receptor-acetylcholine or an analog thereof) IgG-protein A, lectin-carbohydrate, enzyme-enzyme cofactor, enzyme-enzyme-inhibitor, and complementary polynucleotide pairs capable of forming nucleic acid duplexes) and the like.

Furthermore, specific binding pairs can include members that are analogs of the original specific binding members, for example, an analyte-analog. Inmunoreactive specific binding members include antigens, antigen fragments, antibodies and antibody fragments, both monoclonal and polyclonal and complexes thereof, including those formed by recombinant DNA molecules.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

The “analyte” denotes any analyte of interest that is a specific substance or component that is being detected and/or measured by the test strip assays of the present invention. Analytes include but are not limited to biomolecules, organic molecules, small organic molecules, and inorganic compounds capable of being detected by the test strip assays described herein.

The terms “polynucleotide analyte” and “nucleic acid analyte” are used interchangeably and include a single- or double-stranded nucleic acid molecule that contains a target nucleotide sequence. The analyte nucleic acids may be from a variety of sources, e.g., biological fluids or solids, chromosomes, food stuffs, environmental materials, etc., and may be prepared for the hybridization analysis by a variety of means, e.g., proteinase K/SDS, chaotropic salts, or the like.

The term “aptamer” (or nucleic acid antibody) is used herein to refer to a single- or double-stranded DNA or a single-stranded RNA molecule that recognizes and binds to a desired target molecule by virtue of its shape. See, e.g., PCT Publication Nos. WO92/14843, WO91/19813, and WO92/05285, the disclosures of which are incorporated by reference herein. “Polypeptide” and “protein” are used interchangeably herein-and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “semiconductor nanocrystal conjugate” is a semiconductor nanocrystal which is linked to or associated with a specific-binding molecule, as defined above. A “semiconductor nanocrystal conjugate” includes, for example, a semiconductor nanocrystal linked or otherwise associated, through the coat, to a member of a “binding pair” or a “specific-binding molecule” that will selectively bind to a detectable substance present in a sample, e.g., a biological sample as defined herein. The first member of the binding pair linked to the semiconductor nanocrystal can comprise any molecule, or portion of any molecule, that is capable of being linked to a semiconductor nanocrystal and that, when so linked, is capable of recognizing specifically the second member of the binding pair.

The term “antibody” as used herein includes antibodies obtained from both polyclonal and monoclonal preparations, as well as, the following: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monclonal Antibodies and Cancer Therapy, Alan R. Liss, 1985, p. 77.

A semiconductor nanocrystal is “linked” or “conjugated” to, or “associated” with, a specific-binding molecule or member of a binding pair when the semiconductor nanocrystal is chemically coupled to, or associated with the specific-binding molecule. Thus, these terms intend that the semiconductor nanocrystal may either be directly linked to the specific-binding molecule or may be linked via a linker moiety, such as via a chemical linker described below. The terms indicate items that are physically linked by, for example, covalent chemical bonds, physical forces such van der Waals or hydrophobic interactions, encapsulation, embedding, or the like. As an example without limiting the scope of the invention, nanocrystals can be conjugated to molecules that can interact physically with biological compounds such as cells, proteins, nucleic acids, subcellular organelles and other subcellular components. For example, nanocrystals can be associated with biotin which can bind to the proteins, avidin and streptavidin. Also, nanocrystals can be associated with molecules that bind nonspecifically or sequence-specifically to nucleic acids (e.g., DNA and RNA). As examples without limiting the scope of the invention, such molecules include small molecules that bind to the minor groove of DNA (for reviews, see Geierstanger and Wemmer (1995) Ann. Rev. Biophys. Biomol. Struct. 24:463-493; and Baguley (1982) Mol. Cell. Biochem 43:167-181), small molecules that form adducts with DNA and RNA (e.g. CC-1065, see Henderson and Hurley (1996) J. Mol. Recognit. 9:75-87; aflatoxin, see Garner (1998) Mutat. Res. 402:67-75; cisplatin, see Leng and Brabec (1994) IARC Sci. Publ. 125:339-348), molecules that intercalate between the base pairs of DNA (e.g. methidium, propidium, ethidium, porphyrins, etc., for a review see Bailly et al. J. Mol. Recognit. 5:155-171), radiomimetic DNA damaging agents such as bleomycin, neocarzinostatin and other enediynes (for a review, see Povirk (1996) Mutat. Res. 355:71-89), and metal complexes that bind and/or damage nucleic acids through oxidation (e.g. Cu-phenanthroline, see Perrin et al. (1996) Prog. Nucleic Acid Res. Mol. Biol. 52:123-151; Ru(II) and Os(II) complexes, see Moucheron et al. (1997) J. Photochem. Photobiol. B 40:91-106; chemical and photochemical probes of DNA, see Nielsen (1990) J. Mol. Recognit. 3:1-25.

As used herein, a “biological sample” refers to a sample of isolated cells, tissue or fluid, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

A “small molecule” is defined as including an organic or inorganic compound either synthesized in the laboratory or found in nature. Typically, a small molecule is characterized in that it contains several carbon-carbon bonds, and has a molecular weight of less than 1500 grams/Mol.

A “biomolecule” is a synthetic or naturally occurring molecule, such as a protein, amino acid, nucleic acid, nucleotide, carbohydrate, sugar, lipid and the like.

“Optional” or “optionally” means that the subsequently described event or circumstance may or may not occur, and that the description includes instances where the event or circumstance occurs and instances in which it does not. For example, the phrase “optionally overcoated with a shell material” means that the overcoating referred to may or may not be present in order to fall within the scope of the invention, and that the description includes both the presence and absence of such overcoating.

The term “corresponding” is used to denote the reagent or analyte that is capable of specifically binding to a designated analyte or reagent, respectively.

### 2. MODES OF CARRYING OUT THE INVENTION

Before describing the present invention in detail, it is to be understood that this invention is not limited to specific assay formats, materials or reagents, particular formulations or process parameters, as such, of course, may vary. It is also to be understood that the terminology used herein is for the purpose of describing particular embodiments of the invention only, and is not intended to be limiting.

Although a number of compositions, methods, and devices similar or equivalent to those described herein can be used in the practice of the present invention, the preferred materials and methods are described herein.

### 2A. GENERAL OVERVIEW OF THE INVENTION

The present invention is related to immunochromatographic assay methods and to immunochromatographic assay test strip devices, both of which employ semiconductor nanocrystals as a detectable label.

In general, the immunochromatographic test assays of the present invention include several components. First is a test strip comprising a chromatographic medium which is preferably a solid material such as a nitrocellulose membrane; alternately, the strip may be of a non-solid chromatographic medium that is disposed on a solid support such as a polymer or paper sheet. Other suitable materials will be readily apparent to persons of skill in the art.

Disposed on the chromatographic medium at one end of the test strip is a sample reservoir for receiving a test sample suspected of containing one or more analytes of interest. Typically, the sample reservoir is an absorbent pad disposed on the chromatographic medium; however, alternative forms may also be employed, such as a depression or well in the surface of the chromatographic medium into which the test sample may be placed.

Also present is a first detection reagent, which is a conjugate of a first detection ligand, capable of specifically binding a first target moiety of an analyte of interest, and a semiconductor nanocrystal capable of emitting light of a characteristic emission wavelength when exposed to light of a selected excitation wavelength. The nanocrystal-detection ligand conjugate may be in the form of a detection ligand conjugated directly to the nanocrystal; alternately, it may comprise a nanocrystal-dyed microsphere-detection ligand conjugate. In the latter case, microspheres of a wide variety of materials may be employed (see discussion below).

The first detection reagent may be provided in the sample reservoir in dehydrated form; alternately, it may be provided separately, combined with the test sample, and the resulting sample mixture applied to the sample reservoir.

The combination of the test sample and the first detection reagent forms a sample mixture in which, when an analyte of interest is present, the first detection reagent binds the analyte of interest to form a detection complex.

In the case of enhanced assay formats, a second detection reagent will also be provided, as for the first detection reagent, either in the sample reservoir in dehydrated form, or separately, along with the first detection reagent.

The test strips of the present invention further include a capture reagent and a control ligand, both of which are immobilized on the chromatographic medium within a capture region and a control region, respectively, towards the end of the test strip opposite from the sample reservoir and distinct from the sample reservoir and from each other. Depending on the particular assay format, the capture region may be interposed between the sample reservoir and the control region, or vice versa.

The capture reagent includes a capture ligand capable of specifically binding some component of the detection complex to form an immobilized capture complex. The nature of the ligand, and the specific detection complex component to which it binds, will depend on the particular assay format employed.

The control reagent includes a capture ligand capable of specifically binding detection reagent that is unbound to the corresponding analyte of interest.

Both the capture and control reagents are immobilized within the capture and control regions, respectively, of the chromatographic medium. Immobilization may be accomplished by means of chemically binding the reagents to the medium. Alternately, the reagents may be immobilized by means of conjugating them to a microsphere of a size sufficient, relative to the effective pore size of the chromatographic medium, to physically prevent them from moving.

Once the test sample is applied to the sample reservoir, either as is or as part of a sample mixture, the sample mixture is then transported via the chromatographic medium from the sample reservoir towards the capture and control regions. In cases where the analyte of interest is absent from the test sample, the sample mixture will contain only unbound detection reagent, and there will be no detection complex present which may be bound and immobilized by the capture reagent. In such cases, the control ligand immobilized within the control region will bind to and immobilize the unbound detection reagent upon reaching and contacting it. Exposure of the test strip to light of a selected excitation wavelength will cause the nanocrystals conjugated to the detection reagent to emit light of a characteristic wavelength, and a detectable signal will be observed only in the control region, indicating a negative result.

In cases where the analyte of interest is present in the test sample, the sample mixture will contain detection complex in addition to unbound detection reagent. In such cases, the capture region will bind to and immobilize the detection complex upon contacting it, and the control region will bind to and immobilize unbound detection reagent as described above. Exposure of the test strip to light of a selected excitation wavelength will cause the nanocrystals conjugated to the detection reagent to emit light of a characteristic wavelength, and a detectable signal will be observed in both the control region and the capture region, indicating a positive result.

Some embodiments of the test strips of the present invention are capable of detecting multiple analytes of interest present in a single test sample, and further of quantitating the amount of a particular analyte determined to be present (see discussions below).

The test strips of the present invention may be configured according to a number of different assay formats. The two predominant formats are the non-competitive (i.e., direct) and competitive, formats. In the former, the detection ligand and capture ligands each bind different target moieties of the analyte of interest. In the latter, the detection ligand and the control ligand each bind the same target moiety. Direct assay formats include enhanced direct formats such as those employing the biotin/streptavidin binding pair. The nature of the detection, capture and control ligands, and the relative positions of the capture and control regions will vary depending on the assay format (see discussions regarding specific test strip assay embodiments appearing below).

### 2B. DETAILS OF THE TEST STRIP COMPONENTS

Chromatographic Medium: The test strips of the present invention may employ a wide variety of chromatographic media.

The chromatographic material can be any suitably absorbent, porous or capillary possessing material through which a solution containing the analyte can be transported. Natural, synthetic, or naturally occurring materials that are synthetically modified, can be used as the chromatographic material. Such materials include, but not limited to: cellulose materials such as paper, cellulose, and cellulose derivatives such as cellulose acetate and nitrocellulose; fiberglass; cloth, both naturally occurring (e.g., cotton) and synthetic (e.g., nylon); porous gels such as silica gel, agarose, dextran, and gelatin; porous fibrous matrixes; starch based materials, such as SEPHADEX® brand cross-linked dextran chains; ceramic materials; films of polyvinyl chloride and combinations of polyvinyl chloride-silica; and the like.

Typically, a nitrocellulose membrane will be used; other alternatives include but are not limited to cellulose acetate membranes, glass fiber membranes, polyvinylchloride sheets or strips, diazotized paper, polystyrene latex, polyvinylidine fluoride, and nylon membranes. The medium employed may be self-supporting, or may be disposed on the surface of a backing member, which may be made from a wide variety of materials, including but not limited to plastic, paper or glass.

The chromatographic material is one which does not interfere with the production of a detectable signal.

The chromatographic material should have a reasonable inherent strength, or strength can be provided by means of a supplemental support member which may be made from a wide variety of materials, including for example plastic, glass or paper.

A preferred chromatographic material is nitrocellulose. When nitrocellulose is used, however, the material of the sample reservoir should be chosen for its ability to premix the test sample and the first reagent, i.e., fluid-flow through a nitrocellulose membrane is laminar and does not provide the more turbulent flow characteristics which allows the initial mixing of test sample and application pad reagents within the chromatographic material. If nitrocellulose is used as the chromatographic material, then materials such as POREX® hydrophilic polyethylene frit or glass fiber filter paper are appropriate application pad materials because they enable the mixing and reaction of the test sample and application pad reagents within the application pad and before transfer to the chromatographic material.

The particular dimensions of the chromatographic material will be a matter of convenience, depending upon the size of the test sample involved, the assay protocol, the means for detecting and measuring the signal, and the like. For example, the dimensions may be chosen to regulate the rate of fluid migration as well as the amount of test sample to be imbibed by the chromatographic material.

Sample Reservoir: The sample reservoir will optimally be capable of receiving large quantities of test sample, and of releasing the sample into the chromatographic medium at a steady, controlled rate. Toward these ends, a number of materials and configurations may be employed.

For example, the sample reservoir may comprise a depression in the chromatographic medium at one end of the test strip. In a preferred embodiment, the sample reservoir will comprise an absorbent pad, which pad may be made of any of a variety of materials, including but not limited to POREX® hydrophilic polyethylene flit or glass fiber filter paper.

In a preferred embodiment, the first detection reagent (and in the case of enhanced assay formats, the second detection reagent) is present in a dehydrated form within the absorbent pad employed as the sample reservoir (see discussion below).

First Detection Reagent: The first detection reagent comprises semiconductor nanocrystals of varying core sizes (10-150 Å), composition and/or size distribution conjugated to specific-binding molecules which bind specifically to a first target moiety of an analyte of interest. The nanocrystal may be conjugated directly to the binding ligand; alternately, the ligand may be directly conjugated to a nanocrystal-dyed microsphere. In turn, in the latter embodiment, the nanocrystals may be disposed on the external surface of the microsphere, or may alternately be provided within the microsphere's interior.

The first detection reagent may be provided separately from the test strip, and mixed with the test sample to form the sample mixture just prior to performing an analysis; the sample mixture is then applied to the sample reservoir to initiate the test.

Preferably, the first detection reagent may be provided in a dehydrated form in the sample reservoir. Specifically, the first detection reagent may be impregnated and dried in the sample reservoir. Addition of the test sample to the sample reservoir rehydrates the detection reagent and results in formation of the sample mixture in the sample reservoir itself. In turn, binding of the analyte of interest, when present, by the detection reagent occurs, and migration of the sample mixture through the chromatographic medium is initiated.

Detection Ligand: The detection ligand can be any specific binding pair member, for example, an antibody, an immunoreactive fragment of an antibody, and the like. Preferably, the detection ligand is an antibody.

More specifically, the specific-binding molecule may be derived from polyclonal or monoclonal antibody preparations, may be a human antibody, or may be a hybrid or chimeric antibody, such as a humanized antibody, an altered antibody, F(ab′)₂ fragments, F(ab) fragments, Fv fragments, a single-domain antibody, a dimeric or trimeric antibody fragment construct, a minibody, or functional fragments thereof which bind to the analyte of interest. Antibodies are produced using techniques well known to those of skill in the art and disclosed in, for example, U.S. Pat. Nos. 4,011,308; 4,722,890; 4,016,043; 3,876,504; 3,770,380; and 4,372,745.

For example, polyclonal antibodies are generated by immunizing a suitable animal, such as a mouse, rat, rabbit, sheep or goat, with an antigen of interest. In order to enhance immunogenicity, the antigen can be linked to a carrier prior to immunization. Such carriers are well known to those of ordinary skill in the art.

Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler and Milstein (1975) Nature 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Human monoclonal antibodies are obtained by using human rather than murine hybridomas. See, e.g., Cote, et al., Monclonal Antibodies and Cancer Therapy, Alan R. Liss, 1985, p. 77

Monoclonal antibodies or portions thereof may be identified by first screening a B-cell cDNA library for DNA molecules that encode antibodies that specifically bind to p 185, according to the method generally set forth by Huse et al. (1989) Science 246:1275-1281. The DNA molecule may then be cloned and amplified to obtain sequences that encode the antibody (or binding domain) of the desired specificity.

As explained above, antibody fragments which retain the ability to recognize the analyte of interest, will also find use in the subject immunoassays. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy-chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as Fᵥ. See, e.g., Inbar et al. (1972) Proc. Nat. Acad. Sci USA 69:2659-2662; Hochman et al. (1976) Biochem 15:2706-2710; and Ehrlich et al. (1980) Biochem 19:4091-4096.

A single-chain Fv (“sFv” or “scFv”) polypeptide is a covalently linked V_{H}-V_{L} heterodimer which is expressed from a gene fusion including V_{H}- and V_{L}-encoding genes linked by a peptide-encoding linker. Huston et al. (1988) Proc. Nat. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778.

The sFv molecules may be produced using methods described in the art. See, e.g., Huston et al. (1988) Proc. Nat. Acad. Sci. USA 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B: 120-126.

Semiconductor Nanocrystals: The first detection reagent comprises, in addition to the detection ligand discussed above, semiconductor nanocrystals of varying core sizes (10-150 Å), composition and/or size distribution.

Semiconductor nanocrystals for use in the subject methods are made using techniques known in the art. See, e.g., U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. The semiconductor nanocrystals are characterized by their uniform nanometer size.

As discussed above, the selection of the composition of the semiconductor nanocrystal, as well as the size of the semiconductor nanocrystal, affects the characteristic spectral emission wavelength of the semiconductor nanocrystal. Thus, as one of ordinary skill in the art will realize, a particular composition of a semiconductor nanocrystal as listed above will be selected based upon the spectral region being monitored. For example, semiconductor nanocrystals that emit energy in the visible range include, but are not limited to, CdS, CdSe, CdTe, ZnSe, ZnTe, GaP, and GaAs.

Semiconductor nanocrystals that emit energy in the near IR range include, but are not limited to, InP, InAs, InSb, PbS, and PbSe. Finally, semiconductor nanocrystals that emit energy in the blue to near-ultraviolet include, but are not limited to, ZnS and GaN.

For any particular composition selected for the semiconductor nanocrystals to be used in the inventive methods, it is possible to tune the emission to a desired wavelength by controlling the size of the particular composition of the semiconductor nanocrystal. In preferred embodiments, 5-20 discrete emissions (five to twenty different size populations or distributions distinguishable from one another), more preferably 10-15 discrete emissions, are obtained for any particular composition, although one of ordinary skill in the art will realize that fewer than five emissions and more than twenty emissions could be used depending on the monodispersity of the semiconductor nanocrystal particles. If high information density is required, and thus a greater number of distinct emissions, the nanocrystals are preferably substantially monodisperse within the size range given above.

As explained above, “monodisperse,” as that term is used herein, means a colloidal system in which the suspended particles have substantially identical size and shape. In preferred embodiments for high information density applications, monodisperse particles deviate less than 10% rms in diameter, and preferably less than 5%. Monodisperse semiconductor nanocrystals have been described in detail in Murray et al. (1993) J. Am. Chem. Soc. 115:8706, and in Murray, “Synthesis and Characterization of II-VI Quantum Dots and Their Assembly into 3-D Quantum Dot Superlattices,” (1995) Doctoral dissertation, Massachusetts Institute of Technology, which are hereby incorporated by reference in their entireties. One of ordinary skill in the art will also realize that the number of discrete emissions that can be distinctly observed for a given composition depends not only upon the monodispersity of the particles, but also on the deconvolution techniques employed. Semiconductor nanocrystals, unlike dye molecules, can be easily modeled as Gaussians and therefore are more easily and more accurately deconvoluted.

However, for some applications high information density will not be required and it may be more economically attractive to use more polydisperse particles. Thus, for applications that do not require high information density, the linewidth of the emission may be in the range of 40-60 nm.

In a particularly preferred embodiment, the surface of the semiconductor nanocrystal is also modified to enhance the efficiency of the emissions, by adding an overcoating layer to the semiconductor nanocrystal. The overcoating layer is particularly preferred because at the surface of the semiconductor nanocrystal, surface defects can result in traps for electrons or holes that degrade the electrical and optical properties of the semiconductor nanocrystal. An insulating layer at the surface of the semiconductor nanocrystal provides an atomically abrupt jump in the chemical potential at the interface that eliminates energy states that can serve as traps for the electrons and holes. This results in higher efficiency in the luminescent process.

Suitable materials for the overcoating layer include semiconductor materials having a higher bandgap energy than the semiconductor nanocrystal core. In addition to having a bandgap energy greater than the semiconductor nanocrystal core, suitable materials for the overcoating layer should have good conduction and valence band offset with respect to the core semiconductor nanocrystal. Thus, the conduction band is desirably higher and the valence band is desirably lower than those of the core semiconductor nanocrystal. For semiconductor nanocrystal cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet regions may be used. Exemplary materials include ZnS, GaN, and magnesium chalcogenides, e.g., MgS, MgSe, and MgTe. For a semiconductor nanocrystal core that emits in the near IR, materials having a bandgap energy in the visible, such as CdS or CdSe, may also be used. The preparation of a coated semiconductor nanocrystal may be found in, e.g., Dabbousi et al. (1997) J. Phys. Chem. B 101:9463) and Kuno et al. (1997) J. Phys. Chem. 106:9869.

Most semiconductor nanocrystals are prepared in coordinating solvent, such as trioctylphosphine oxide (TOPO) and trioctyl phosphine (TOP) resulting in the formation of a passivating organic layer on the nanocrystal surface comprised of the organic solvent. This layer is present on semiconductor nanocrystals containing an overcoating and those that do not contain an overcoating. Thus, either of these classes of passivated semiconductor nanocrystals is readily soluble in organic solvents, such as toluene, chloroform and hexane. As one of ordinary skill in the art will realize, these functional moieties may be readily displaced or modified to provide an outer coating that renders the semiconductor nanocrystals suitable for use as the detectable labels of the present invention, as described further below. Furthermore, based upon the desired application, a portion of the semiconductor nanocrystal functionality, or the entire surface of the semiconductor nanocrystal functionality may b& modified by a displacement reaction, based upon the desired use therefor.

After selection of the composition of semiconductor nanocrystal for the desired range of spectral emission and selection of a desired surface functionalization compatible with the system of interest, it may also be desirable to select the minimum number of semiconductor nanocrystals needed to observe a distinct and unique spectral emission of sufficient intensity for spectral identification. Selection criteria important in determining the minimum number of semiconductor nanocrystals needed to observe a distinct and unique spectral emission of sufficient intensity include providing a sufficient number of semiconductor nanocrystals that are bright (i.e., that emit light versus those that are dark) and providing a sufficient number of semiconductor nanocrystals to average out over the blinking effect observed in single semiconductor nanocrystal emissions. Nirmal et al., (1996) Nature 383:802.

For example, eight or more semiconductor nanocrystals of a particular composition and particle size distribution can be provided. If, for example, the desired method of use utilizes three different particle size distributions of a particular composition, eight of each of the three different particle size distributions of a semiconductor nanocrystal is used, in order to observe sufficiently intense spectral emissions from each to provide reliable information regarding the location or identity of a particular analyte of interest. One of ordinary skill in the art will realize, however, that fewer than eight semiconductor nanocrystals of a particular composition and particle size distribution may be utilized provided that a unique spectral emission of sufficient intensity is observed, as determined by the selection criteria set forth above.

The above method can be used to prepare separate populations of semiconductor nanocrystals, wherein each population exhibits a different characteristic photoluminescence spectrum. Each of a plurality of populations of semiconductor nanocrystals can be conjugated to distinct first members of binding pairs for use in a multiplexed assay or analytical method in which each of a plurality of corresponding second members of the binding pairs can be detected simultaneously.

The narrow spectral linewidths and nearly gaussian symmetrical lineshapes lacking a tailing region observed for the emission spectra of nanocrystals combined with the tunability of the emission wavelengths of nanocrystals allows high spectral resolution in a system with multiple nanocrystals. In theory up to 10-20 or more different-sized nanocrystals or different size distributions of monodisperse populations of nanocrystals from different preparations of nanocrystals, with each sample having a different emission spectrum, can be used simultaneously in one system, i.e., multiplexing, with the overlapping spectra easily resolved using techniques well known in the art, e.g., optically with or without the use of deconvolution software.

As discussed previously, the ability of the semiconductor nanocrystals to produce discrete optical transitions, along with the ability to vary the intensity of these optical transitions, enables the development of a versatile and dense encoding scheme. The characteristic emissions produced by one or more sizes of semiconductor nanocrystals attached to, associated with, or embedded within a particular support, compound or matter enables the identification of the analyte of interest and/or its location. For example, by providing N sizes of semiconductor nanocrystals (each having a discrete optical transition), each having M distinguishable states resulting from the absence of the semiconductor nanocrystal, or from different intensities resulting from a particular discrete optical transition, Mⁿ different states can be uniquely defined. In the case wherein M is 2, in which the two states could be the presence or absence of the semiconductor nanocrystal, the encoding scheme would thus be defined by a base 2 or binary code. In the case wherein M is 3, in which the three states could be the presence of a semiconductor nanocrystal at two distinguishable intensities and its absence, the encoding scheme would be defined by a base 3 code. Herein, such base M codes wherein M is greater than 2 are termed higher order codes. The advantage of higher order codes over a binary order code is that fewer identifiers are required to encode the same quantity of information.

As one of ordinary skill in the art will realize, the ability to develop a higher order encoding system is dependent upon the number of different intensities capable of detection by both the hardware and the software utilized in the decoding system. In particularly preferred embodiments, each discrete emission or color, is capable of being detectable at two to twenty different intensities. In a particularly preferred embodiment wherein ten different intensities are available, it is possible to employ a base 11 code comprising the absence of the semiconductor nanocrystal, or the detection of the semiconductor nanocrystal at 10 different intensities.

Clearly, the advantages of the semiconductor nanocrystals, namely the ability to observe discrete optical transitions at a plurality of intensities, provides a powerful and dense encoding scheme that can be employed in a variety of disciplines. In general, one or more semiconductor nanocrystals may act as a barcode, wherein each of the one or more semiconductor nanocrystals produces a distinct emissions spectrum. These characteristic emissions can be observed as colors, if in the visible region of the spectrum, or may also be decoded to provide information about the particular wavelength at which the discrete transition is observed. Likewise, for semiconductor nanocrystals producing emissions in the infrared or ultraviolet regions, the characteristic wavelengths that the discrete optical transitions occur at provide information about the identity of the particular semiconductor nanocrystal, and hence about the identity of or location of the analyte of interest.

The color of light produced by a particular size, size distribution and/or composition of a semiconductor nanocrystal can be readily calculated or measured by methods which will be apparent to those skilled in the art. As an example of these measurement techniques, the bandgaps for nanocrystals of CdSe of sizes ranging from 12 Å to 115 Å are given in Murray et al. (1993) J. Am. Chem. Soc. 115:8706. These techniques allow ready calculation of an appropriate size, size distribution and/or composition of semiconductor nanocrystals and choice of excitation light source to produce a nanocrystal capable of emitting light device of any desired wavelength. An example of a specific system for automated detection for use with the present methods includes, but is not limited to, an imaging scheme comprising an excitation source, a spectrometer (or any device capable of spectrally resolving the image, or a set of narrow band filters) and a detector array. In one embodiment, the apparatus consists of a blue or UV source of light, of a wavelength shorter than that of the luminescence detected. This may be a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm), a HeCd laser; solid state diode lasers in the blue such as GaN and GaAs (doubled) based lasers or the doubled or tripled output of YAG or YLF based lasers; or any of the pulsed lasers with output in the blue, to name a few.

The luminescence from the dots may be passed through an imaging subtracting double monochromator (or two single monochromators with the second one reversed from the first), for example, consisting of two gratings or prisms and a slit between the two gratings or prisms. The monochromators or gratings or prisms can also be replaced with a computer controlled color filter wheel where each filter is a narrow band filter centered at the wavelength of emission of one of the dots. The monochromator assembly has more flexibility because any color can be chosen as the center wavelength. Furthermore, a CCD camera or some other two dimensional detector records the images, and software color codes that image to the wavelength chosen above. The system then moves the gratings to a new color and repeats the process. As a result of this process, a set of images of the same spatial region is obtained and each is color-coded to a particular wavelength that is needed to analyze the data rapidly.

In another embodiment, the apparatus is a scanning system as opposed to the above imaging scheme. In a scanning scheme, the sample to be analyzed is scanned with respect to a microscope objective. The luminescence is put through a single monochromator or a grating or prism to spectrally resolve the colors. The detector is a diode array that then records the colors that are emitted at a particular spatial position. The software then ultimately recreates the scanned image and decodes it.

Microspheres: In a number of embodiments, the test strip assays of the present invention will employ first detection reagents, capture reagents and/or control reagents wherein the detection, capture and/or control ligands, respectively, are conjugated to a support, such as microspheres dyed with semiconductor nanocrystals. Supports useful in the practice of the invention may be of a variety of sizes and may be made from a variety of materials. In addition, they may be colored so as to allow for visualization without the use of specialized detection apparatuses.

For example, a wide variety of materials are useful for preparing microspheres employed by the present invention. The particles can be selected by one skilled in the art from any suitable type of particulate material composed of polystyrene, polymethylacrylate, polyacrylamide, polypropylene, latex, polytetrafluoroethylene, polyacrylonitrile, polycarbonate, glass or similar materials.

Microparticle size will affect the flow rate of the detection reagent and/or detection complex through the chromatographic medium. Optimal flow rate is achieved by choosing microspheres that are no greater than 1/10 of the effective pore size of the medium employed.

Both the capture and the control reagents may be conjugated to microspheres as a means of physically immobilizing those reagents within the capture and control regions (see discussion below). In such cases, microsphere size is chosen relative to the effective pore size of the chromatographic medium such that migration through the chromatographic medium is prevented.

Production of Nanocrystal Conjugates: Semiconductor nanocrystal-ligand conjugates or semiconductor nanocrystal-dyed microsphere-ligand conjugates are manufactured using techniques well known in the art. See, e.g. U.S. Pat. No. 5,990,479, U.S. Pat. No. 5,284,752, and U.S. Pat. No. 5,326,692.

The present invention uses a composition comprising semiconductor nanocrystals associated with a specific-binding molecule or affinity molecule, such that the composition can detect the presence and/or amounts of biological and chemical compounds, detect interactions in biological systems, detect biological processes, detect alterations in biological processes, or detect alterations in the structure of biological compounds. Without limitation, semiconductor nanocrystal conjugates comprise any molecule or molecular complex, linked to a semiconductor nanocrystal, that can interact with a biological target, to detect biological processes, or reactions, as well as alter biological molecules or processes. Preferably, the molecules or molecular complexes or conjugates physically interact with a biological compound. Preferably, the interactions are specific. The interactions can be, but are not limited to, covalent, noncovalent, hydrophobic, hydrophilic, electrostatic, van der Waals, or magnetic. Preferably, these molecules are small molecules, proteins, or nucleic acids or combinations thereof.

Semiconductor nanocrystal conjugates can be made using techniques known in the art. For example, moieties such as TOPO and TOP, generally used in the production of semiconductor nanocrystals, as well as other moieties, may be readily displaced and replaced with other functional moieties, including, but not limited to carboxylic acids, amines, aldehydes, and styrene to name a few. One of ordinary skill in the art will realize that factors relevant to the success of a particular displacement reaction include the concentration of the replacement moiety, temperature and reactivity. Thus, for the purposes of the present invention, any functional moiety may be utilized that is capable of displacing an existing functional moiety to provide a semiconductor nanocrystal with a modified functionality for a specific use.

The ability to utilize a general displacement reaction to modify selectively the surface functionality of the semiconductor nanocrystals enables functionalization for specific uses. For example, because detection of biological compounds is most preferably carried out in aqueous media, a preferred embodiment of the present invention utilizes semiconductor nanocrystals that are solubilized in water. In the case of water-soluble semiconductor nanocrystals, the outer layer includes a compound having at least one linking moiety that attaches to the surface of the particle and that terminates in at least one hydrophilic moiety. The linking and hydrophilic moieties are spanned by a hydrophobic region sufficient to prevent charge transfer across the region. The hydrophobic region also provides a “pseudo-hydrophobic” environment for the nanocrystal and thereby shields it from aqueous surroundings. The hydrophilic moiety may be a polar or charged (positive or negative) group. The polarity or charge of the group provides the necessary hydrophilic interactions with water to provide stable solutions or suspensions of the semiconductor nanocrystal. Exemplary hydrophilic groups include polar groups such as hydroxides (—OH), amines, polyethers, such as polyethylene glycol and the like, as well as charged groups, such as carboxylates (—CO²—), sulfonates (SO³⁻), phosphates (—PO₄²⁻ and PO₃²⁻), nitrates, ammonium salts (—NH⁴⁺), and the like. A water-solubilizing layer is found at the outer surface of the overcoating layer. Methods for rendering semiconductor nanocrystals water-soluble are known in the art and described in, e.g., International Publication No. WO 00/17655, published Mar. 30, 2000.

The affinity for the nanocrystal surface promotes coordination of the linking moiety to the semiconductor nanocrystal outer surface and the moiety with affinity for the aqueous medium stabilizes the semiconductor nanocrystal suspension.

A displacement reaction may be employed to modify the semiconductor nanocrystal to improve the solubility in a particular organic solvent. For example, if it is desired to associate the semiconductor nanocrystals with a particular solvent or liquid, such as pyridine, the surface can be specifically modified with pyridine or pyridine-like moieties to ensure solvation.

The surface layer may also be modified by displacement to render the semiconductor nanocrystal reactive for a particular coupling reaction. For example, displacement of TOPO moieties with a group containing a carboxylic acid moiety enables the reaction of the modified semiconductor nanocrystals with amine containing moieties (commonly found on solid support units) to provide an amide linkage. Additional modifications can also be made such that the semiconductor nanocrystal can be associated with almost any solid support. A solid support, for the purposes of this invention, is defined as an insoluble material to which compounds are attached, such as a detection ligand or a microsphere.

For example, the semiconductor nanocrystals of the present invention can readily be functionalized to create styrene or acrylate moieties, thus enabling the incorporation of the semiconductor nanocrystals into, or adsorption onto, microspheres made from polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose, and the like.

For a detailed description of these linking reactions, see, e.g., U.S. Pat. No. 5,990,479; Bruchez et. al. (1998) Science 281:2013-2016., Chan et. al. (1998) Science 281:2016-2018, Bruchez “Luminescent Semiconductor Nanocrystals: Intermittent Behavior and use as Fluorescent Biological Probes” (1998) Doctoral dissertation, University of California, Berkeley, and Mikulec “Semiconductor Nanocrystal Colloids: Manganese Doped Cadmium Selenide, (Core)Shell Composites for Biological Labeling, and Highly Fluorescent Cadmium Telluride” (1999) Doctoral dissertation, Massachusetts Institute of Technology.

Capture Reagent: Generally, the capture reagent comprises a binding pair member that is capable of specifically binding the detection complex. The test strips of the present invention typically employ an antibody specific for a second epitope of the analyte of interest as the capture reagent, although other binding pair members may also be employed. The specific nature of the capture reagent will depend on the assay format employed.

Enhanced Sandwich Assay Components: In test strips of the present invention which employ the enhanced sandwich assay format, a second detection reagent comprises a second detection ligand which binds a second target moiety of the analyte of interest, concomitantly with the first detection reagent binding a first target moiety, to form the detection complex. The capture ligand in enhanced sandwich assays then specifically binds a component of the second detection reagent in order to immobilize the detection complex within the capture region.

The second detection ligand and the capture ligand in enhanced sandwich assays may be selected from among a variety of binding pairs. For example, in one embodiment, the second detection reagent comprises a bioatinylated monoclonal antibody and the capture reagent comprises streptavidin which specifically binds the biotin moieties of the second detection reagent in order to immobilize the detection complex. Other embodiments which make use of binding pairs other than biotin/streptavidin (see the discussion above) are also contemplated.

Control Reagents and Control Ligands: The specific nature of the control ligand component of the control reagent will depend on the assay format employed.

In the case of direct sandwich assays and enhanced sandwich assays, the control reagent may comprise a binding pair member capable of specifically binding the first detection reagent. In such cases the control ligand, as for the first and second detection ligands and the capture ligands, are preferably an antibody, and may be derived from polyclonal or monoclonal antibody preparations, may be a human antibody, or may be a hybrid or chimeric antibody, such as a humanied antibody, an altered antibody, F(ab′)₂ fragments, F(ab) fragments, Fv fragments, a single-domain antibody, a dimeric or trimeric antibody fragment construct, a minibody, or functional fragments thereof which bind to the analyte of interest. Antibodies are produced using techniques well known to those of skill in the art. See the discussion above.

In the case of competitive binding assays, the control reagent will comprise the first target moiety of the analyte of interest, as well as fragments and analogs thereof which retain the binding characteristics of the first target moiety.

As an additional positive control, the test strips of the present invention may further include an independent control conjugate comprising a nanocrystal-first binding pair member conjugate or a nanocrystal-dyed microsphere-first binding pair member conjugate which is provided either in the sample reservoir or as a component of the first detection reagent when that reagent is provided separately.

The emission peak of the nanocrystal label incorporated into the independent control conjugate is distinct from that exhibited by the nanocrystals of the first detection reagent.

The embodiments which employ an independent control conjugate will also include an independent control reagent, which will comprise the second binding pair member immobilized on the chromatographic region in, for example, a separate independent control region which is distinct from the original control region; other locations are also possible. Binding of the independent control reagent to the second binding pair member immobilized in the independent control region and subsequent detection serves as a positive control indicating successful migration of material from the sample reservoir through the chromatographic medium.

Immobilization of the Capture and Control Reagents on the Chromatographic Medium: The capture and control reagents employed in the test strip assays of the present invention may be immobilized on the chromatographic medium in a variety of ways. For example, those reagents may be directly or indirectly attached to the chromatographic material. Direct attachment methods include adsorption, absorption and covalent binding such as by use of (i) a cyanogen halide, e.g., cyanogen bromide or (ii) by use of glutaraldehyde. Depending on the assay, it may be preferred, however, to retain or immobilize the desired reagent on the chromatographic material indirectly through the use of insoluble microparticles to which the reagent has been attached. The means of attaching a reagent to the microparticles encompasses both covalent and non-covalent means, that is adhered, absorbed or adsorbed. By “retained and immobilized” is meant that the particles, once on the chromatographic material, are not capable of substantial movement to positions elsewhere within the material.

Miscellaneous Additional Components: The test strip assays of the present invention may also include additional components such as a plastic housing or case which encloses and protects the test strip, as well as a foil pouch to protect and store the final product. A dessicant, useful in order to keep the test strips dry during storage before use, can either be added into the foil pouch or incorporated directly into the sample reservoir.

As discussed above, the chromatographic medium may be disposed on a support member in order to support that medium and/or, in cases where the medium used is a material such as a nitrocellulose membrane, to give support and added strength. The medium may be attached to the support member either by means of an adhesive or by a direct casting process, in which the medium is formed on and adheres to the support member.

### 2C. MULTIPLE ANALYTE STRIPS

The test strips of the present invention may be configured so as to be capable of detection of multiple analytes.

For example, a plurality of detection reagents, each specific for a single analyte of interest, may be employed. The detection complexes formed by the binding of each detection reagent to the corresponding analyte of interest may then be detected and distinguished by a variety of means. For example, each detection reagent may be associated with a nanocrystal having a distinct emission peak; alternately, each of the detection complexes may be distinguished by means of capture reagents specific for each detection complex immobilized at distinct locations along the chromatographic medium.

### 2D. TEST STRIP MANUFACTURE

Immunochromatographic test strips are produced using conventional materials according to standard protocols for this type of assay. See, e.g., U.S. Pat. No. 4,703,017.

### 2E. DETECTION SYSTEMS

An advantage of the test strips of the present invention is that the amount of detection complex that binds to the capture region may be quantified, and the amount of analyte present in the test sample determined therefrom.

An excitation light source is provided, wherein this excitation source has significant light output at a wavelength lower than the emission peak of the semiconductor nanocrystal used in the first detection reagent. Suitable sources include light emitting diodes of various types, lamps of various types, lasers of various types, and the like.

The results of a typical strip test using semiconductor nanocrystal or semiconductor nanocrystal-dyed microsphere can be determined by visual inspection, i.e., by exposing the strip to a light source having appropriate emission characteristics and noting the presence and/or absence of luminescence at appropriate test and/or control regions on the strip.

A mechanism for collecting signal generated by the semiconductor nanocrystal is also provided, wherein the signal collection mechanism collects light emitted from the area suspected of containing semiconductor nanocrystals. This emission light can be projected onto a variety of detectors that convert incoming light into an electronic signal including a photodiode, a photomultiplier device, a charge-coupled diode, or the like. An assay in which more than one color of semiconductor nanocrystal or semiconductor nanocrystal-dyed microsphere also involves the use of techniques to resolve spectrally the emitted light. Examples of techniques for resolving spectra of light emitted by semiconductor nanocrystals include, but are not limited to: (1) the use of bandpass filters placed in front of the detector, or moving different bandpass filters in front of the detector to detect sequentially each specific semiconductor nanocrystal emission; (2) multiple detectors, each with a separate distinct bandpass filter coupled with means to split the emitted light into those sensors; (3) a scanning approach can be used where the sample is moved to be detected sequentially by different detection devices; and (4) the use of multiple detectors, for example, a linear CCD, coupled with a spectral dispersion device, for example, a grating or prism, wherein each element in the linear CCD then detects light of a narrow wavelength range.

Detection of the control strip can be carried out either simultaneously with detection of the test region using multiple detectors or sequentially by moving the sample relative to the detectors. The magnitude of the signal detected for each semiconductor nanocrystal species is analyzed to determine the amount of analyte present, depending on the assay format.

### 2F. EXEMPLARY EMBODIMENTS OF THE PRESENT INVENTION

A variety of specific embodiments of the present invention described above are possible.

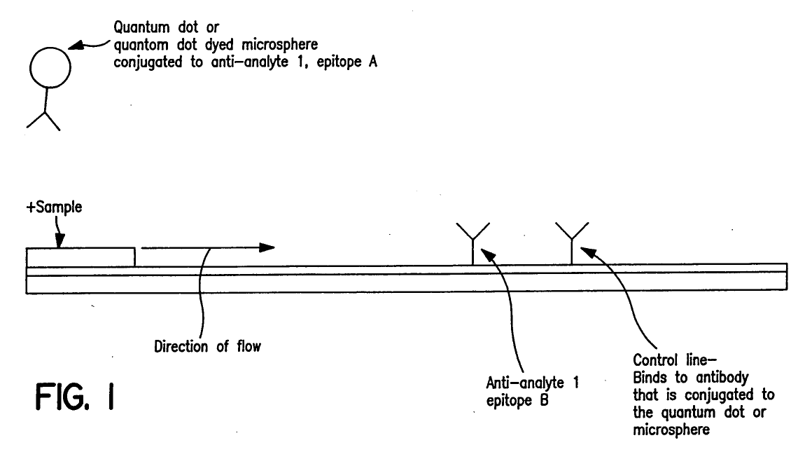

In a first exemplary embodiment, illustrated in FIG. 1, a semiconductor nanocrystal or a microsphere spectrally dyed with semiconductor nanocrystals is conjugated to a detection ligand, comprising an analyte-specific antibody that recognizes a first epitope on a single analyte of interest, to form a detection reagent. The semiconductor nanocrystal associated with the detection reagent exhibits a distinct emission characteristic.

The capture region is a zone of immobilized capture reagent comprising an antibody that is specific for a second epitope of the analyte. The control region is a zone of immobilized control reagent comprising a species-specific antibody that recognize the detection ligand.

The detection reagent described above is either pre-adsorbed on the sample pad or mixed with the analyte sample prior to the test. The chromatographic strip assay is initiated by placing the test sample or the sample mixture, respectively, on the sample pad. If analyte is present in the sample, it binds to the detection reagent to form a detection complex and the test region captures this complex. The control region captures any unbound detection reagent and any excess detection complex. Once the sample has passed the test and control regions the assay result is determined by measuring the luminescent emission of the semiconductor nanocrystal-conjugates or semiconductor nanocrystal-dyed microsphere-conjugate in the capture region.

Measurement and quantitation are carried out using a detection scheme as described above.

In a second exemplary embodiment, a semiconductor nanocrystal or a microsphere dyed with a semiconductor nanocrystal is conjugated to a detection ligand, comprising an analyte-specific antibody that recognizes a first distinct epitope on an analyte of interest, to form a detection reagent. Multiple detection reagents, each specific for a single analyte of interest, may be present. Each different analyte-specific antibody is conjugated to a semiconductor nanocrystal or semiconductor nanocrystal-dyed microsphere exhibiting an emission characteristic distinct from that of the other detection reagents.

The capture region in this second exemplary embodiment comprises multiple zones of capture reagents, each zone containing an immobilized capture reagent comprising an antibody specific for a second epitope on a specific analyte. Zones are separated by enough membrane to allow clear distinction of the bands. The control zone comprises a zone of immobilized species-specific antibody that recognizes the antibodies which comprise the detection ligands of the detection reagents.

Detection reagents and a test sample suspected of containing one or more analytes of interest are added to the sample pad thus initiating the sample flow along the membrane. Each analyte of interest present in the test sample binds to the corresponding detection reagent to form a detection complex. Each detection complex is captured by the corresponding test region. Each different detection complex is captured by its specific capture reagent. Each of the control regions captures any unbound detection reagent and any excess detection complex.

Once the sample passes the capture and control regions the test result can be determined by measuring the emission luminescence of the detection complexes in the capture regions. Measurement and quantitation can be carried out using detection as described above.

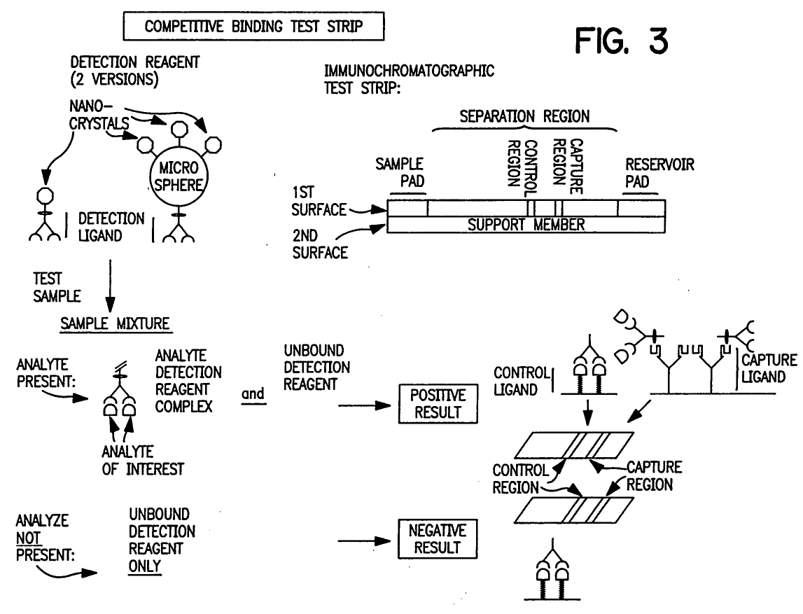

In a third exemplary embodiment, illustrated in FIG. 3, a semiconductor nanocrystal or a microsphere spectrally dyed with a semiconductor nanocrystal is conjugated to a detection ligand, comprising an analyte-specific antibody that recognizes a first distinct epitope on the analyte, to form a detection reagent. There may be multiple detection reagents. The detection ligand of each is conjugated to a semiconductor nanocrystal or semiconductor nanocrystal-dyed microsphere exhibiting a distinct emission characteristic.

The capture region is a zone of capture reagent comprising immobilized analyte. There may be multiple capture regions, each comprising a particular analyte of interest. The control region is a zone of control reagent comprising species-specific antibody that recognizes the antibody which comprises the detection ligand. Conjugates and analyte sample are added to the sample pad thus initiating the chromatographic strip assay.

If analyte is present in the sample it binds to a detection reagent and blocks the ability of the immobilized analyte comprising the capture reagent to bind. In such cases, the control region does not capture any detection reagent. If only an undetectable amount or no analyte is present, the detection reagent binding sites are not blocked and hence the detection complex is bound by the control region.

The capture region binds any bound or unbound detection reagent. Once the sample passes the test and control regions the test result can be determined by measuring the emission luminescence of the semiconductor nanocrystal-conjugates or semiconductor nanocrystal-dyed microsphere-conjugate in the capture region.

Measurement and quantitation are carried out using the detection system as described above.

A fourth embodiment invention finds particular utility when it is difficult to initiate sample flow due to high protein binding to hydrophobic membranes. In this embodiment, an inert material that cannot bind protein is used as the chromatographic medium.

A semiconductor nanocrystal or a microsphere spectrally dyed with semiconductor nanocrystals is conjugated to a detection ligand, comprising an analyte-specific antibody that recognizes a distinct epitope of a particular analyte of interest, to form a detection reagent. There may be multiple detection reagents. The detection ligand of each detection reagent is conjugated to a semiconductor nanocrystal or semiconductor nanocrystal dyed microsphere with a distinct emission characteristic.

The capture regions are multiple zones (or a multiplexed single zone) of immobilized capture reagents comprising antibodies conjugated to very large microspheres, e.g., microspheres that are not substantially smaller than the pore size of the substrate membrane of the strip. The microspheres are held in place in the membrane by physical forces.

The control region is a zone of immobilized control reagents comprising species-specific antibodies that are attached to large microspheres, which antibodies are capable of recognizing the antibodies that are conjugated to the detection reagents.

Detection reagents and a test sample suspected of containing one or more analytes of interest are added to the sample pad thus initiating the sample flow along the inert chromatographic medium.

Analyte present in the sample binds to the detection reagent and this complex is captured by the test region. Each different analyte-detection conjugate complex is captured by its specific second antibody.

The control region captures any unbound detection reagent and any excess detection complex.

Once the sample has passed the test and control regions the test result can be determined by measuring the emission luminescence of the semiconductor nanocrystal-conjugates or semiconductor nanocrystal dyed microsphere-conjugates in the test region.

Measurement and quantitation are carried out using a detection system as described above.

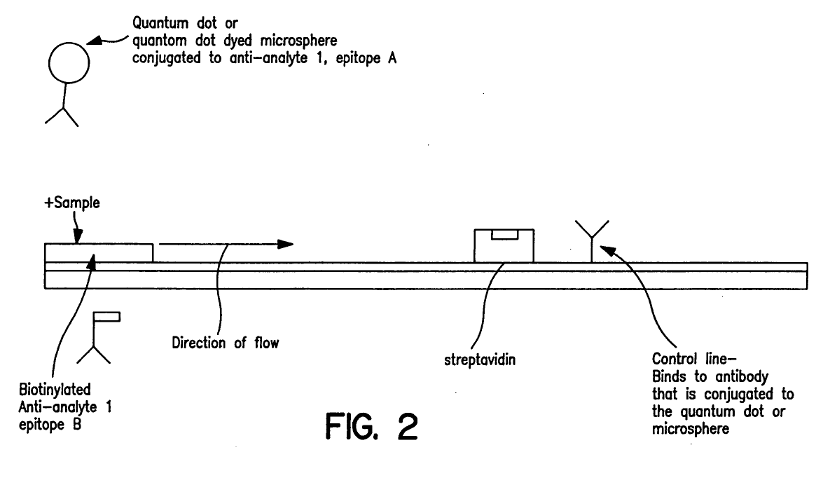

A fifth exemplary embodiment of the invention, illustrated in FIG. 2, finds particular utility when use of a specific binding molecule other than an antibody is required to detect an analyte.

A semiconductor nanocrystal or a microsphere spectrally dyed with semiconductor nanocrystals is conjugated to a first detection ligand, comprising an analyte-specific antibody that recognizes a first distinct epitope of an analyte of interest, to form a first detection reagent. There may be multiple first detection reagents, the detection ligand of each being capable of specifically binding with a distinct analyte of interest. Each of the first detection reagents comprise a semiconductor nanocrystal or a semiconductor nanocrystal dyed microsphere with a distinct emission characteristic, conjugated to the first detection ligand.

For each first detection regent which binds a particular analyte of interest, a second detection reagent, comprising an antibody that specifically recognizes a second epitope of the same analyte is also present. The second detection reagent further comprises a binding pair member, such as biotin, conjugated to the antibody.

The capture region comprises a zone of immobilized capture reagent comprising a second binding pair member capable of specifically binding to the first binding pair member conjugated to each of the second detection ligands. The control region comprises a zone of immobilized control reagent comprising antibody or biomolecule that specifically recognizes the detection reagent in the presence or absence of analyte.

Detection reagents and a test sample suspected of containing one or more analytes of interest are added to the sample pad thus initiating the chromatographic strip assay. Analyte present in the sample binds to the corresponding detection reagent to form a detection complex. The detection complex is captured by the capture region; The control region captures any unbound detection reagent and any excess detection complex.

Once the sample has passed the test and control regions the test result can be determined by measuring the emission luminescence of the semiconductor nanocrystal-conjugates or semiconductor nanocrystal dyed microsphere-conjugates in the test region.

Measurement and quantitation are carried out using the detection system as described above.

### 3. APPLICATIONS

Immunochromatographic test strips which utilize semiconductor nanocrystals as a detectable label possess several useful advantages over test strips employing other detectable labels that are currently known in the art.

For example, semiconductor nanocrystals allow quantitative results to be generated in terms of the luminescence intensity of the detection conjugates trapped in the test region. The tunable emission properties of semiconductor nanocrystals allows multiple analyte detection to be carried out in one strip test. The use of semiconductor nanocrystals further allows a permanent record of assay results to be generated and archived. In addition, due to the intensity of the emission from semiconductor nanocrystals, greater sensitivity and dynamic range can be achieved relative to conventional detection reagents. Furthermore, the ability to tune not only the excitation wavelength but also the emission wavelength allows the design of a system in which substrate autofluorescence is minimized by selecting well-separated excitation and emission wavelengths.

These advantages have utility in assays such as, but not limited to, drug testing panels where multiple analyte detection, identification and/or quantitation are desirable. Diagnostic tests for pathogenic infections and multiple pollutant screens also benefit from the multiple analyte capabilities and quantitative nature of semiconductor nanocrystals employed as detection reagents in immunochromatographic test strip assays.

With respect to diagnostic tests, the immunochromatographic test strip assays of the present invention are useful for diagnosing a variety of human conditions and diseases on the basis of the presence of certain antigens which accompany them. Examples include but are not limited to: pregnancy (hCG present in urine); Down's syndrome (α-fetoprotein and acetylcholinesterase in amniotic fluid); myocardial infarction (cardiac markers such as troponin-T and myoglobin); sexually transmitted diseases including gonorrhea, chlamydia, and syphilis (antigens associated with each organism present in blood and/or semen or vaginal secretions); hepatitis, including HAV, HBV and HCV (viral antigens characteristic of each type of virus present in blood, urine, and other bodily fluids); and HIV (viral antigens present in blood, urine and other bodily fluids).

In the context of veterinary diagnostic tests, a variety of diseases having substantial economic impact are caused by bacteria that are present in the environment, transmitted by other animals, or both. A non-limiting example is bovine mastitis, a disease resulting from infection of the mammary glands by streptococcus bacteria. A variety of streptococcus species, each possessing characteristic surface antigens, may cause mastitis. Hence, the test strips of the present invention may be configured so as to detect the presence of those antigens and thereby permit diagnosis and appropriate treatment of the particular species causing the infection, as well as providing information regarding whether the infection is more likely attributable to environmental contaminants or infectious transmission from other infected animals.

Test strip assays capable of screening samples for multiple pollutants are another application of the present invention. For example, water samples may be screened for the presence of pathogenic bacteria (e.g., vibrio cholerae, helicobacter, cyclospora, toxoplasma, microsporidium, acanthamoeba, and Cyanobacteria (blue-green algae)), viruses (e.g., hepatitis and others), and parasites.

As an alternative strategy, the test strip assays of the present invention may be configured such that they are capable of detecting nucleic acid analytes. Such assays may be used as diagnostic tests or screens which detect and/or quantify pathogenic bacteria, viruses, and other microorganisms, as well as detecting the existence of a variety of physiological conditions, based on the presence of certain characteristic nucleic acids.

In yet another exemplary embodiment, the test strip assays of the present invention may be designed to detect and quantify a variety of biomolecules in addition to those discussed above, including but not limited to the following: hormones, pheromones, growth factors, immunoglobulins of a particular antigen specificity and/or of a particular idiotype, enzymes, metabolic substrates and products, and other biomolecules which are indicative of a physiological or pathological condition of interest and which are capable of being specifically bound by a second molecule which is amenable to being used in the construction of a detection agent and a capture and/or control reagent.

### EXPERIMENTAL

The following example is intended to illustrate, but not limit, this invention.

Efforts have been made to ensure accuracy with respect to numbers used (e.g, amounts, temperatures, etc.), but some experimental error and deviation should, of course, be allowed for.

### Example 1

### Binding of 563 nm Emission Semiconductor Nanocrystals to the Capture Region of a Nitrocellulose Strip

Detection complexes localized in the capture area of a nitrocellulose test strip are capable of generating a fluorescence signal of an intensity that significantly exceeds background.

10 μl of a 1 μM solution of semiconductor nanocrystals was suspended in PBS and 250 μl was applied to the reagent pad of a nitrocellulose membrane (10 μM pore size) having streptavidin immobilized in the capture zone (Roche Diagnostics, Indianapolis, Ind.). The nanocrystals were allowed to wick through the nitrocellulose and become non-specifically bound to the streptavidin capture line. The accumulation was observed using a ZEISS® 25CFL fluorescent microscope (Carl Zeiss, Germany).

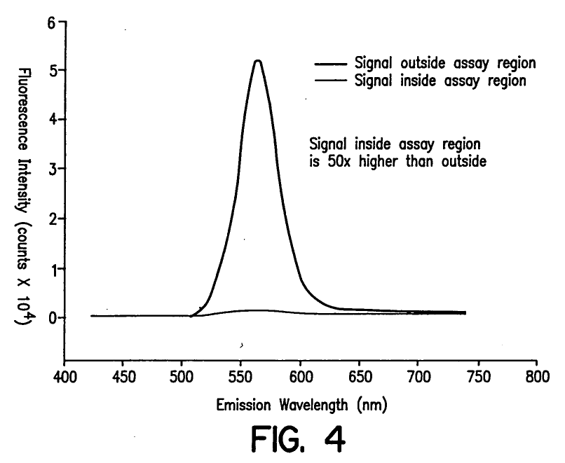

The amount of non-specifically bound nanocrystals present at the capture line was then quantified by-exciting the capture line and the surrounding nitrocellulose membrane using a 488 nm argon ion laser (Coherent, Santa Clara, Calif.). The emitted light was detected through a microscope objective (20X, Edmunds Scientific, Barrington, N.J.) attached to an Oceanoptics specrophotometer (El Dorado Hills, Calif.). The results are shown in FIG. 4.

Thus, novel methods for using semiconductor nanocrystals are disclosed. Although preferred embodiments of the subject invention have been described in some detail, it is understood that obvious variations can be made without departing from the spirit and the scope of the invention as defined by the appended claims.

1. A method for determining the presence, amount or presence and amount of an analyte of in a test sample, said method comprising the steps of:
(I) applying the test sample to a test strip to form a sample mixture in a sample reservoir, said test strip comprising
(A) a chromatographic medium;
(B) the sample reservoir disposed on said chromatographic medium for receiving said test sample, said sample reservoir comprising
(i) a first detection reagent comprising
(a) a first detection ligand capable of selectively binding a first target moiety of said analyte of interest, wherein (i) said first detection ligand is conjugated with a semiconductor nanocrystal or a microsphere comprising a semiconductor nanocrystal which, when exposed to a light of a selected excitation wavelength, is capable of emitting light of a characteristic emission spectrum, and (ii) binding of said first detection ligand to said first target moiety forms a detection complex,
(C) a capture reagent immobilized on said chromatographic medium within a capture region which is distinct from said sample reservoir, wherein said capture reagent comprises a capture ligand capable of selectively binding said first detection complex to form an immobilized capture complex; and
(D) a control ligand immobilized on said chromatographic medium within a control region distinct from said sample reservoir and said capture region, wherein said control ligand is capable of selectively binding said first detection ligand to form an immobilized control complex; wherein (i) said test strip has first and second ends, said sample reservoir is disposed at said first end, and said capture region is interposed between said sample reservoir and said control region, (ii) said sample mixture comprises said test sample and said first detection reagent, (iii) said sample mixture is transported via said chromatographic medium from said first to said second end, (iv) said first detection ligand binds said first target moiety to form said detection complex, said detection complex is bound by said capture reagent, and said first detection ligand which is not bound to said first target moiety is bound to said control ligand; and
(II) exposing said test strip to said light of a selected excitation wavelength, wherein the production of light of said characteristic emission peak in both the capture and control regions is indicative of the presence of the analyte in the test sample.

2-4. (canceled)

5. The method of claim 1, wherein one or more of said detection reagents comprises said semiconductor nanocrystal conjugated directly to said detection ligand.

6. The method of claim 1, wherein one or more of said detection reagents comprises said microsphere conjugated directly to said detection ligand.

7-9. (canceled)

10. The method of claim 1, wherein said first detection ligand is an antibody.

11-12. (canceled)

13. The method of claim 1, wherein said first detection ligand is a nucleic acid molecule.

14-16. (canceled)

17. The method of claim 1, wherein said capture ligand is selected from the group consisting of a nucleic acid molecule, biotin and an antibody.

18-20. (canceled)

21. The method of claim 1, wherein said control ligand is selected from the group consisting of a nucleic acid molecule, biotin and an antibody.

22. A method for determining the presence, amount or presence and amount of an analyte of interest in a test sample, said method comprising the steps of:
(I) applying the test sample to a test strip to form a sample mixture in a sample reservoir, said test strip comprising
(A) a chromatographic medium;
(B) the sample reservoir disposed on said chromatographic medium for receiving said test sample, said sample reservoir comprising
(i) a first detection reagent, comprising
(a) a first detection ligand capable of selectively binding a first target moiety of said analyte of interest,
wherein said first detection ligand is conjugated with a semiconductor nanocrystal or a microsphere comprising a semiconductor nanocrystal which, when exposed to a light of a selected excitation wavelength, is capable of emitting light of a characteristic emission spectrum, and
(ii) a second detection reagent comprising a second detection ligand capable of selectively binding (a) a second target moiety of said analyte of interest, and (b) a capture ligand,
wherein binding of said first detection ligand to said first target moiety and said second detection ligand to said second target moiety forms a detection complex,
(C) a capture reagent immobilized on said chromatographic medium within a capture region which is distinct from said sample reservoir, wherein said capture reagent comprises a capture ligand capable of selectively binding said second detection ligand to form an immobilized capture complex; and
(D) a control ligand immobilized on said chromatographic medium within a control region distinct from said sample reservoir and said capture region, wherein said control ligand is capable of selectively binding said first detection ligand not bound to said first target moiety to form an immobilized control complex;
wherein (i) said test strip has first and second ends, said sample reservoir is disposed at said first end, and said control region is interposed between said sample reservoir and said capture region, (ii) said sample mixture comprises said test sample and said first detection reagent, (iii) said sample mixture is transported via said chromatographic medium from said first to said second end, (iv) said first detection ligand binds said first target moiety and said second detection ligand binds said second target moiety to form said detection complex, (v) said detection complex is bound by said capture reagent, and (vi) said first detection ligand which is not bound to said first target moiety is bound to said control ligand; and
(II) exposing said test strip to said light of a selected excitation wavelength, wherein the production of light of said characteristic emission peak in both the capture and control regions is indicative of the presence of the analyte in the test sample.

23-26. (canceled)

27. The method of claim 22, wherein one or more of said detection reagents comprises said microsphere conjugated directly to said detection ligand.

28-33. (canceled)

34. The method of claim 22, wherein said first detection ligand is selected from the group consisting of a nucleic acid molecule, biotin and an antibody.

35-37. (canceled)

38. The method of claim 22, wherein said capture ligand is selected from the group consisting of a nucleic acid molecule, biotin and an antibody.

39-41. (canceled)

42. The method of claim 22, wherein said control ligand is selected from the group consisting of a nucleic acid molecule, biotin and an antibody.

43-45. (canceled)

46. The method of claim 22, wherein said second detection ligand is selected from the group consisting of a nucleic acid molecule, biotin and an antibody.

47-49. (canceled)

50. A method for determining the presences, amount or presence and amount of an analyte of interest in a test sample, said method comprising the steps of:
(I) applying the test sample to a test strip to form a sample mixture in a sample reservoir, said test strip comprising
(A) a chromatographic medium;
(B) the sample reservoir disposed on said chromatographic medium for receiving said test sample, said sample reservoir comprising
(i) a first detection reagent comprising
(a) a first detection ligand capable of selectively binding a first target moiety of said analyte of interest, wherein (i) said first detection ligand is conjugated with a semiconductor nanocrystal or a microsphere comprising a semiconductor nanocrystal which, when exposed to a light of a selected excitation wavelength, is capable of emitting light of a characteristic emission peak, and (ii) binding of said first detection ligand to said first target moiety forms a first detection complex,
(C) a capture reagent immobilized on said chromatographic medium within a capture region which is distinct from said sample reservoir, wherein said capture reagent comprises a capture ligand capable of selectively binding said first detection complex to form an immobilized capture complex; and
(D) a control reagent immobilized on said chromatographic medium within a control region distinct from said sample reservoir and said capture region, wherein said control ligand is capable of selectively binding said first detection ligand to form an immobilized control complex;
wherein (i) said test strip has first and second ends, said sample reservoir is disposed at said first end, and said capture region is interposed between said sample reservoir and said control region,(ii) said sample mixture comprises said test sample and said first detection reagent,(iii) said sample mixture is transported via said chromatographic medium from said first to said second end,(iv) said first detection ligand binds said first target moiety and said second detection ligand binds said second target moiety to form said detection complex, (v) said detection complex is bound by said capture reagent, and (vi) said first detection ligand which is not bound to said first target moiety is bound to said control ligand; and
(II) exposing said test strip to said light of a selected excitation wavelength, wherein the production of light of said characteristic emission peak in both the capture and control regions is indicative of the presence of the analyte in the test sample.

51-53. (canceled)

54. The method of claim 50, wherein one or more of said detection reagents comprises said semiconductor nanocrystal conjugated directly to said detection ligand.

55. The method of claim 50, wherein one or more of said detection reagents comprises said microsphere conjugated directly to said detection ligand.

56-61. (canceled)

62. The method of claim 50, wherein said first detection ligand is selected from the group consisting of a nucleic acid molecule, biotin and an antibody.

63-65. (canceled)

66. The method of claim 50, wherein said capture ligand is selected from the group consisting of a nucleic acid molecule, biotin and an antibody.

67-69. (canceled)

70. The method of claim 50, wherein said control ligand is selected from the group consisting of a nucleic acid molecule, biotin and an antibody.

71. A test strip for determining the presence, amount or presence and amount of an analyte of interest suspected of being present in a test sample, comprising:
(I) a chromatographic medium;
(II) a sample reservoir disposed on said chromatographic medium for receiving said test sample, said sample reservoir comprising
(A) a first detection reagent comprising
(i) a first detection ligand capable of selectively binding a first target moiety of said analyte of interest, wherein (a) said first detection ligand is conjugated with a semiconductor nanocrystal or a microsphere comprising a semiconductor nanocrystal which, when exposed to a light of a selected excitation wavelength, is capable of emitting light of a characteristic emission peak, and (b) binding of said first detection ligand to said first target moiety forms a first detection complex,
(III) a capture reagent immobilized on said chromatographic medium within a capture region which is distinct from said sample reservoir, wherein said capture reagent comprises a capture ligand capable of selectively binding said first detection complex to form an immobilized capture complex; and
(IV) a control ligand immobilized on said chromatographic medium within a control region distinct from said sample reservoir and said capture region, wherein said control ligand is capable of selectively binding said first detection ligand to form an immobilized control complex.

72-73. (canceled)

74. The test strip of claim 71, wherein one or more of said detection reagents comprises said semiconductor nanocrystal conjugated directly to said detection ligand.

75. The test strip of claim 71, wherein one or more of said detection reagents comprises a microsphere conjugated directly to said detection ligand, wherein said microsphere is dyed with said semiconductor nanocrystals.

76-81. (canceled)

82. The test strip of claim 71, wherein said first detection ligand is selected from the group consisting of a nucleic acid molecule, biotin and an antibody.

83-85. (canceled)

86. The test strip of claim 71, wherein said capture ligand is selected from the group consisting of a nucleic acid molecule, biotin and an antibody.

87-89. (canceled)

90. The test strip of claim 71, wherein said control ligand is selected from the group consisting of a nucleic acid molecule, biotin and an antibody.

91. The test strip of claim 71, wherein said test strip has first and second ends, said sample reservoir is disposed at said first end, and said capture region is interposed between said sample reservoir and said control region, and wherein further said capture ligand is capable of binding a second target moiety of the analyte, and said control ligand is capable of binding said first detection ligand which is not bound to said first target moiety.

92. The test strip of claim 71, wherein said test strip has first and second ends, said sample reservoir is disposed at said first end, and said control region is interposed between said sample reservoir and said capture region, and wherein said control ligand is capable of binding said first detection ligand which is not bound to said first target moiety, and said capture ligand is capable of binding said first detection complex.

93. The test strip of claim 92, wherein said control ligand comprises said analyte.

94. The test strip of claim 92, wherein said control ligand comprises said first target moiety of said analyte.

95. A test strip for determining the presence, amount or presence and amount of an analyte of interest suspected of being present in a test sample, comprising:
(I) a chromatographic medium;
(II) a sample reservoir disposed on said chromatographic medium for receiving said test sample, said sample reservoir comprising
(A) a first detection reagent comprising
(i) a first detection ligand capable of selectively binding a first target moiety of said analyte of interest, wherein said first detection ligand is conjugated with a semiconductor nanocrystal or a microsphere comprising a semiconductor nanocrystal which, when exposed to a light of a selected excitation wavelength, is capable of emitting light of a characteristic emission peak,
(B) a second detection reagent comprising
(i) a second detection ligand capable of selectively binding (a) a second target moiety of said analyte of interest, and (b) a capture ligand,
(III) a capture reagent immobilized on said chromatographic medium within a capture region which is distinct from said sample reservoir, wherein said capture reagent comprises a capture ligand capable of selectively binding said second detection ligand to form an immobilized capture complex; and
(IV) a control ligand immobilized on said chromatographic medium within a control region distinct from said sample reservoir and said capture region, wherein said control ligand is capable of selectively binding said first detection ligand not bound to said first target moiety to form an immobilized control complex, wherein said test strip has first and second ends, said sample reservoir is disposed at said first end, and said capture region is interposed between said sample reservoir and said control region.

96-98. (canceled)

99. The test strip of claim 95, wherein said second detection ligand is selected from the group consisting of a nucleic acid molecule and an antibody.

100. The test strip of claim 95, wherein said second detection ligand is a biotinylated antibody and said capture ligand is streptavidin or avidin.

101-102. (canceled)
